(19) United States
(12) Patent Application Publication
(10) Pub. No.: US 2009/0076334 A1
(43) Pub. Date: Mar. 19, 2009

(54) Illuminated Vaginal Speculum and Illumination Device

(76) Inventor: Tien-Sheng Chen, Taipei City (TW)

Correspondence Address: KAMRATH & ASSOCIATES P.A., 4825 OLSON MEMORIAL HIGHWAY, SUITE 245, GOLDEN VALLEY, MN, 55422, US

(21) Appl. No.: 11/934,299

(22) Filed: Nov. 2, 2007

(30) Foreign Application Priority Data
Sep. 19, 2007 (TW) 096215769

(51) Int. Cl. A61B 1/32

(52) U.S. Cl. 600/223

(57) ABSTRACT

This invention relates to an illuminated vaginal speculum suitable for cervical speculoscopy to enable a user to distinguish abnormal cells from normal cells, the illuminated vaginal speculum comprising: a first blade having a first open part and a first holding part; a second blade, which is pivoted to the first blade, comprising a second open part and a second holding part; an adjuster installed between the first and the second blades for adjusting the relative angle between the first and the second open parts; and a first illumination member connected to the first open part or the second open part, the first illumination member being capable of emitting light of a color different from that of the normal cells so as to enable the user to distinguish abnormal cells from normal cells.

### BACKGROUND OF THE INVENTION

1. Field of the Invention

The present invention relates to an illuminated vaginal speculum; more particularly, it relates to an illuminated vaginal speculum suitable for cervical colposcopy to enable a user to distinguish abnormal cells from normal cells.

2. Description of the Related Art

A vaginal speculum is an instrument designed for insertion into the vagina to facilitate inspection or medication. During an examination conducted with a vaginal speculum, a light source is needed to illuminate the inside of the vagina. Traditionally, an external light source is used, such as a handheld light projector or a head-wearable light. The external light source, due to inconvenience and poor illumination effects, creates several problems when used.

To provide more desirable illumination means, several solutions have been proposed in the related art. For example, in U.S. patent No. US2003/0176772, a disposable vaginal speculum having a light device is disclosed. However, an improvement in the light sources is rarely discussed.

In Taiwan, cervical cancer is one of the most common cancers in women. Conventionally, the major diagnosis of the cervical cancer is the cervical smear test, a technique known to have a rather high false negative rate. Due to this high false negative rate, some patients with normal test results are sometimes found to be in early cancer stage after a visual examination with a vaginal colposcopy.

In order to increase the accuracy of cervical cancer screening, a popular technique called PAPSURE™ is often used in conjunction with the cervical smear test. Research shows that the accuracy can be increased to more than 90% by the combination of the two tests.

Usually, the PAPSURE™ is performed by the following steps. First, 3-5% diluted acetic acid is smeared on the cervix with a cotton swab, wherein the potential pathological area may turn into opaque white plaques, commonly known as “acetate white”; then a fluorescent bar is bent and fixed at the front end of a vaginal speculum for observation of the color of the cervix.

Due to the often troublesome procedure mentioned above, an improvement of the vaginal speculum for facilitating the cervical screening is desirable.

### SUMMARY OF THE INVENTION

It is therefore an object of the present invention to provide a novel illuminated vaginal speculum suitable for being used in PAPSURE™ so as to enable users to distinguish abnormal cells from normal cells easily.

It is another object of the present invention to provide an illuminated vaginal speculum capable of emitting light of a color different from that of the normal cells and enhancing the contrast of cells of different colors so as to enable users to distinguish abnormal cells from normal cells. The wavelength of the light emitted by the illuminated vaginal speculum, without limitation, is preferably less than that of red visible light, such as yellow light, green light, blue light, purple light, etc. More preferably, the illuminated vaginal speculum may emit green light, which is complementary to the red light reflected by normal cells, so that users may distinguish abnormal cells from normal cells more easily.

It is still another object of the present invention to provide an illuminated vaginal speculum having two illumination members. These two members may be switched by the user to fit the needs arising in different circumstances.

It is yet another object of the present invention to provide an illumination device compatible with a vaginal speculum. The illumination device may be mounted on the vaginal speculum without an illumination member so as to increase the utility of the vaginal speculum.

To achieve the objects mentioned above, this invention provides an illuminated vaginal speculum suitable for cervical colposcopy to enable a user to distinguish abnormal cells from normal cells, the illuminated vaginal speculum comprising: a first blade having a first open part and a first holding part; a second blade, which is pivoted to the first blade, comprising a second open part and a second holding part; an adjuster installed between the first and the second blades for adjusting the relative angle between the first and the second open parts; and a first illumination member connected to the first open part or the second open part, the first illumination member being capable of emitting light of a color different from that of the normal cells so as to enable the user to distinguish abnormal cells from normal cells.

In addition, the present invention provides an illumination device mountable on a vaginal speculum in order to enable a user to distinguish abnormal cells from normal cells in a cervical colposcopy screening, the illumination device comprising an illumination member and a connection part, wherein the illumination member is capable of emitting light of a color different from that of the normal cells so as to enable the user to distinguish abnormal cells from normal cells, and the connection part is capable of mounting the illumination member on the vaginal speculum.

Other objects, advantages, and novel features of the invention will become more apparent from the following detailed description when taken in conjunction with the accompanying drawings.

### BRIEF DESCRIPTION OF THE DRAWINGS

These and other objects and advantages of the present invention will become apparent from the following description of the accompanying drawings, which disclose several embodiments of the present invention. It is to be understood that the drawings are to be used for purposes of illustration only, and not as a definition of the invention.

In the drawings, wherein similar reference numerals denote similar elements throughout the several views:

FIG. 1 is an illustration of one embodiment of the illuminated vaginal speculum of this invention.

FIG. 2 is a side view of one embodiment of the illuminated vaginal speculum of this invention.

FIG. 3 is a side view of one embodiment of the illuminated vaginal speculum of this invention.

FIG. 4 is an illustration of another embodiment of the illuminated vaginal speculum of this invention.

FIG. 5 is an illustration of one embodiment of the illumination device of this invention.

### DETAILED DESCRIPTION OF THE PREFERRED EMBODIMENT

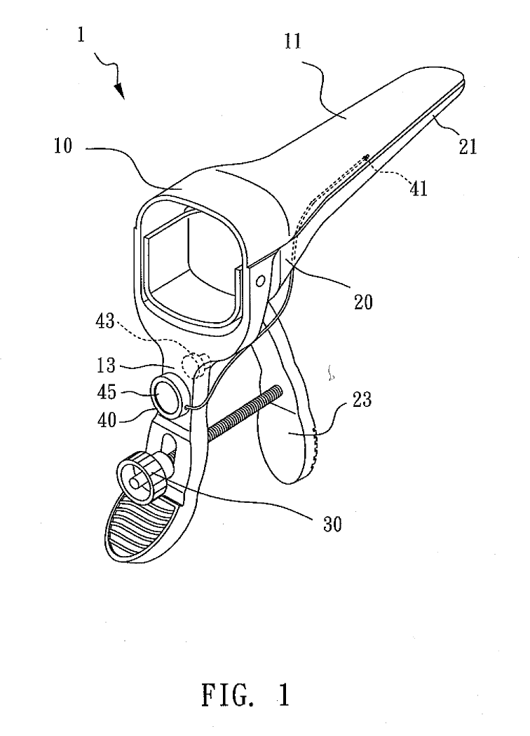

Please refer to FIG. 1 for an illustration of one embodiment of the illuminated vaginal speculum 1 of this invention. The illuminated vaginal speculum 1 of this invention is suitable for cervical colposcopy to enable a user to distinguish abnormal cells from normal cells. The illuminated vaginal speculum 1 mainly comprises: a first blade 10 having a first open part 11 and a first holding part 13; a second blade 20, which is pivoted to the first blade 10, comprising a second open part 21 and a second holding part 23; an adjuster 30 installed between the first and the second blades 10, 20 for adjusting the relative angle between the first and the second open parts 11,21; and a first illumination member 40 connected to the second open part 21, the first illumination member 40 being capable of emitting light of a color different from that of the normal cells so as to enable the user to distinguish abnormal cells from normal cells.

In this embodiment, the first illumination member 40 is connected to the second open part 21; however, the first illumination member 40 may also be connected to the first open part 11, depending on the user's purpose.

In most cases, the first illumination member 40 may comprise without limitation a battery 45, a light switch 43, and a light emitting diode (LED) light bulb 41. For example, the first illumination member 40 may also be connected to an external power source via an electrical wire and have no battery 45. Moreover, the first illumination member 40, such as a fluorescent bar, may also be built into the first open part 11 or the second open part 21. In addition, as shown in FIG. 1, the electrical wire of the first illumination member 40 may be embedded in the first blade 10 or the second blade 20 so as to increase its utility.

In most situations, the first illumination member 40 may emit light of a color different from that of the normal cells, preferably green light, which is complementary to the red light reflected by normal cells, so that users may distinguish abnormal cells from normal cells more easily.

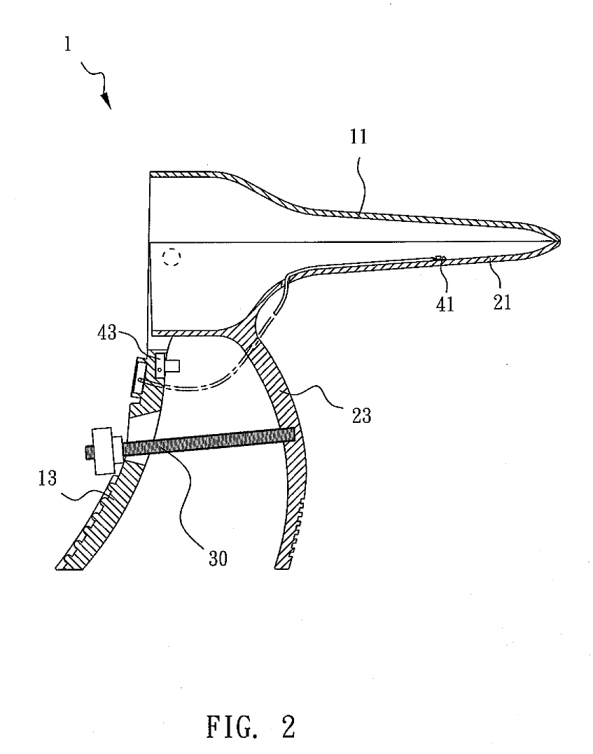

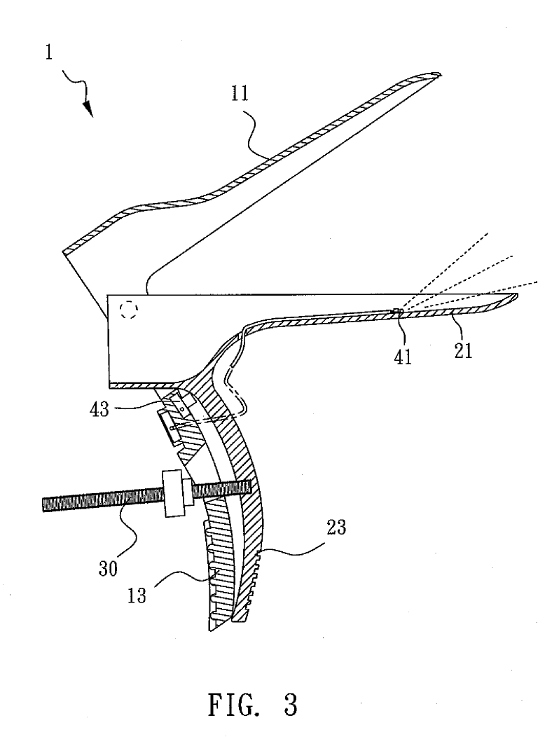

In most situations, the wavelength of the light emitted by the illuminated vaginal speculum 1, without limitation, is preferably less than that of red visible light, such as yellow light, green light, blue light, purple light, etc. In this invention, the red light refers to the visible light having a wavelength ranging substantially from 650 to 750 nm. Accordingly, any visible light having a wavelength less than 650 nm may enable users to distinguish abnormal cells from normal cells. Preferably, the illuminated vaginal speculum 1 may emit visible light with a wavelength ranging substantially from 500 to 600 nm. Please also refer to FIGS. 2 and 3 for side views of one embodiment of the illuminated vaginal speculum 1 of this invention. In FIG. 2, when the first open part 11 and the second open part 12 are closed, the gap between the first holding part 13 and the second holding part 23 is relatively large, and the light switch 43 is off because the LED light bulb 41 is not electrically conducted. When the illuminated vaginal speculum 1 is used, as shown in FIG. 3, the first open part 11 and the second open part 12 are opened, and the light switch 43 is on, subsequently turning on the LED light bulb 41.

With the design mentioned above, users may automatically turn on the LED light bulb 41 when they use the illuminated vaginal speculum 1; similarly, the power supply for the LED light bulb 41 may be turned off automatically when the first open part 11 and the second open part 12 are closed.

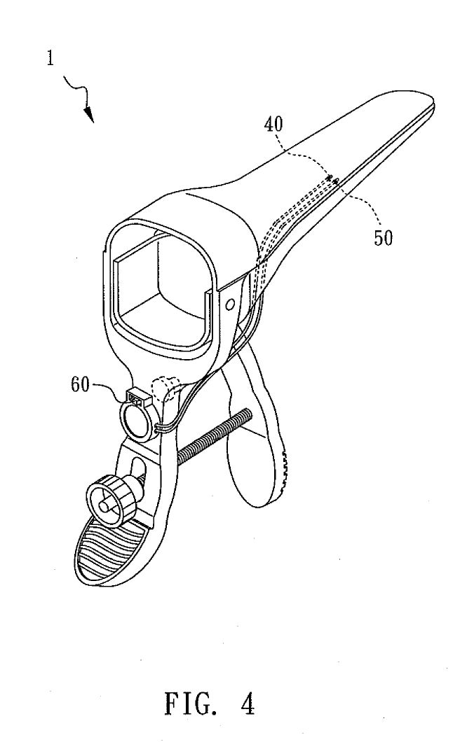

Refer to FIG. 4 for an illustration of another embodiment of the illuminated vaginal speculum 1 of this invention. As shown by FIG. 3, the illuminated vaginal speculum 1 of this invention has a first illumination member 40 and a second illumination member 50, both of which may share one power supply and switch. The first illumination member 40 and the second illumination member 50, which emit lights of different colors, may be mounted on the same open part or on different open parts. In addition, the illuminated vaginal speculum 1 may further comprise a switch device 60 which enables users to selectively turn on the first illumination member 40 or the second illumination member 50, largely increasing the utility of the invention. Accordingly, the illuminated vaginal speculum 1 of this invention may be applied to both PAPSURE™ and ordinary cervical observation.

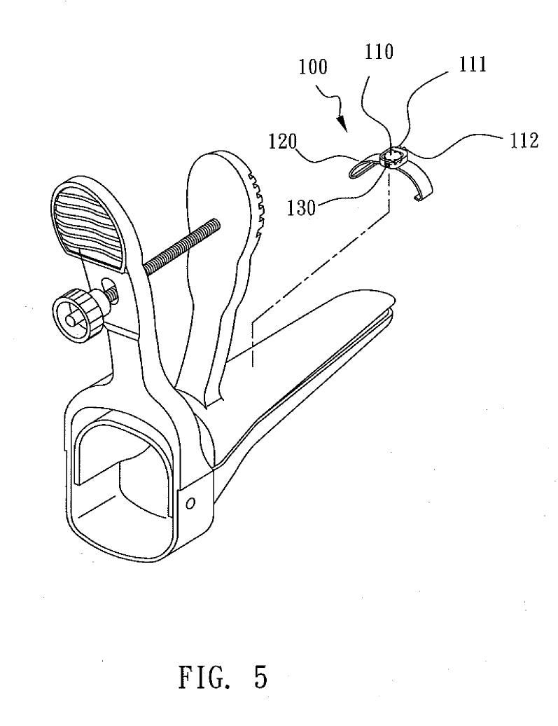

Finally, please refer to FIG. 5 for an illustration of one embodiment of the illumination device 100 of this invention. In order to provide a traditional vaginal speculum with a light source, this invention discloses an illumination device 100 mountable on the traditional vaginal speculum to enable a user to distinguish abnormal cells from normal cells in a cervical speculoscopy screening. The illumination device 100 comprises an illumination member 110 and a connection part 120, wherein the illumination member 110 is capable of emitting light of a color different from that reflected by normal cells so as to enable the user to distinguish abnormal cells from normal cells, and the connection part 120 is capable of mounting the illumination member on the vaginal speculum.

In this invention, the connection part 120 may be a buckling belt, as shown in this embodiment, or any other means for mounting the illumination member 110 on the vaginal speculum, such as without limitation adhesive, a locking member, and so on.

Meanwhile, to provide a smaller illumination member 110 with adequate luminance, the illumination member 110 may comprise a first LED light bulb 111 and a second LED light bulb 112. The second LED light bulb 112 may emit light of a color different from that of the first LED light bulb 111. By the application of a switch member 130, users may selectively turn on the first LED light bulb 111 or the second LED light bulb 112 so as to provide the light source that they desire.

Although the present invention has been explained in relation to its preferred embodiments, it is to be understood that many other possible modifications and variations can be made without departing from the spirit and scope of the invention as hereinafter claimed.

What is claimed is:

1. An illuminated vaginal speculum suitable for cervical colposcopy to enable a user to distinguish abnormal cells from normal cells, the illuminated vaginal speculum comprising:
a first blade having a first open part and a first holding part;
a second blade, which is pivoted to the first blade, comprising a second open part and a second holding part;
an adjuster installed between the first and the second blades for adjusting the relative angle between the first and the second open parts; and
a first illumination member connected to the first open part or the second open part, the first illumination member being capable of emitting light of a color different from that of the normal cells so as to enable the user to distinguish abnormal cells from normal cells.

2. The illuminated vaginal speculum as claimed in claim 1, wherein the first illumination member comprises a light emitting diode light bulb.

3. The illuminated vaginal speculum as claimed in claim 1, wherein the first illumination member further comprises a light switch installed on the first holding part or the second holding part for turning on the light emitting diode light bulb when the first open part and the second open part are opened.

4. The illuminated vaginal speculum as claimed in claim 1, wherein the first illumination member is capable of emitting green light.

5. The illuminated vaginal speculum as claimed in claim 1, wherein the first illumination member is capable of emitting blue light.

6. The illuminated vaginal speculum as claimed in claim 1, wherein the first illumination member is capable of emitting yellow light.

7. The illuminated vaginal speculum as claimed in claim 1, wherein the first illumination member is capable of emitting purple light.

8. The illuminated vaginal speculum as claimed in claim 1, wherein the first illumination member is capable of emitting visible light with a wavelength substantially less than 650 nm.

9. The illuminated vaginal speculum as claimed in claim 1, wherein the first illumination member is capable of emitting visible light with a wavelength ranging substantially from 500 nm to 600 nm.

10. The illuminated vaginal speculum as claimed in claim 1, further comprising a second illumination member connected to the first open part or the second open part, the second illumination member being capable of emitting light of a color different from that of the first illumination member.

11. The illuminated vaginal speculum as claimed in claim 10, further comprising a switch device that enables the user to switch between the first and the second illumination members.

12. An illumination device mountable on a vaginal speculum to enable a user to distinguish abnormal cells from normal cells in a cervical colposcopy screening, the illumination device comprising an illumination member and a connection part, wherein the illumination member is capable of emitting light of a color different from that of the normal cells so as to enable the user to distinguish abnormal cells from normal cells, and the connection part is capable of mounting the illumination member on the vaginal speculum.

13. The illumination device as claimed in claim 12, wherein the illumination member comprises a first light emitting diode light bulb.

14. The illumination device as claimed in claim 13, wherein the illumination member further comprises a second light emitting diode light bulb that emits light of a color different from that of the first light emitting diode light bulb.

15. The illumination device as claimed in claim 12, wherein the illumination member is capable of emitting green light.

16. The illumination device as claimed in claim 12, wherein the illumination member is capable of emitting blue light.

17. The illumination device as claimed in claim 12, wherein the illumination member is capable of emitting yellow light.

18. The illumination device as claimed in claim 12, wherein the illumination member is capable of emitting visible light with a wavelength substantially less than 650 nm.

19. The illumination device as claimed in claim 12, wherein the illumination member is capable of emitting visible light with a wavelength ranging substantially from 500 nm to 600 nm.

20. The illumination device as claimed in claim 14, further comprising a switch member for enabling the user to switch between the first and the second light emitting diode light bulbs.
